(19) United States
(12) Patent Application Publication
(10) Pub. No.: US 2020/0155753 A1
(43) Pub. Date: May 21, 2020

(54) ANTI-REFLUX ENEMA BUCKET SYSTEM WITH PULLEY RESTRICTOR

(71) Applicant: Ningbo Albert Novosino Co., Ltd, Ningbo (CN)

(72) Inventors: Yonggui Zhang, Ningbo (CN); Haibo Hu, Ningbo (CN)

(21) Appl. No.: 16/674,061

(22) Filed: Nov. 5, 2019

(30) Foreign Application Priority Data
Nov. 19, 2018 (CN) 201821901978.5

(51) Int. Cl. A61M 3/02

(52) U.S. Cl. CPC A61M 3/0287; A61M 3/0216; A61M 3/0233

(57) ABSTRACT

Disclosed are various embodiments for an anti-reflux enema bucket system that is leakproof, easy to disassemble and clean, prevents reflux, has components that can be independently cleaned and sanitized, and controls a rate at which water is expelled from a nozzle outlet. In various embodiments, the enema bucket system includes an enema bucket having an opening at a top for filling the enema bucket with solution, a nozzle comprising a nozzle outlet, an anti-reflux coupler positioned between the enema bucket and the nozzle through which the solution passes from the enema bucket to the nozzle outlet; and tubing fluidly coupling the enema bucket, the anti-reflux coupler, and the nozzle. A pulley restrictor may selectively control a speed at which the solution is introduced into the nozzle and expelled from the nozzle outlet.

### CROSS-REFERENCE TO RELATED APPLICATION

This application claims the benefit of and priority to Chinese Patent Application No. 201821901978.5 filed Nov. 19, 2018, now published as Chinese Patent Publication No. 209430902 U, the contents of which being incorporated in their entirety herein.

### TECHNICAL FIELD

This invention relates to the technical field of enemas and, in particular, describes a washable anti-reflux enema bucket system having a pulley restrictor and an anti-reflux coupler.

### BACKGROUND

Enemas include devices that can be utilized by patients and medical practitioners for cleansing the body, such as vaginal, anal, and other bodily cavities. Generally, enemas include plastic bodies filled with a solution, such as clean water, which is injected into a cavity of a person through use of a syringe-type device. During this process, the solution may “reflux,” where some solution is returned into a tube positioned between an enema bag or enema bulb, and a syringe device, thereby contaminating the solution and affecting the use of conventional enemas. In some instances, the reflux can return to the enema and contaminate the solution residing therein.

Various types of enemas can be suspended or hung to create natural water pressure that can be used in applying a desirable flow of solution. However, with existing enemas, such as enema bags, an opening for filling solution in the enema bag is intended to be small to prevent solution from spilling out during use, which makes filling the enema with solution difficult and inconvenient. Further, existing enemas have switches or valves that do not completely stop solution from leaking when turned off, which also leads to a buildup of dirt or other debris. Even further, various components of the enemas cannot be sterilized using, for example, boiling water, and cannot be disassembled for cleaning.

### BRIEF SUMMARY OF INVENTION

Disclosed are various embodiments for an enema bucket system that is leakproof, easy to disassemble and clean, prevents reflux, has components that can be independently cleaned and sanitized, and controls a rate at which water is expelled from a nozzle outlet.

In various embodiments, the enema bucket system includes an enema bucket having an opening at a top for filling the enema bucket with solution, a nozzle comprising a nozzle outlet, an anti-reflux coupler positioned between the enema bucket and the nozzle through which the solution passes from the enema bucket to the nozzle outlet; and tubing fluidly coupling the enema bucket, the anti-reflux coupler, and the nozzle.

The anti-reflux coupler can include an inlet for receiving at least a portion of the solution from the enema bucket; an outlet for expelling the solution into the nozzle; and a check valve configured to prevent reflux of the solution from the nozzle. The check valve may include one of an umbrella valve; a duckbill valve; a slit-cutting valve; and a flapper valve.

In some embodiments, the enema bucket system further includes a pulley restrictor configured to selectively control a speed at which the solution is introduced into the nozzle and expelled from the nozzle outlet. The pulley restrictor can include a body, a guide, and a wheel movably coupled to the guide, the body comprising a first end and a second end. The tubing can be positioned through the body of the pulley restrictor such that the wheel is positioned near the tubing. An adjustment of the wheel along the guide to the first end of the body causes pressure to be applied to the tubing and restricts flow of the solution therein. An adjustment of the wheel along the guide to the second end of the body relieves the pressure on the tubing and permits the flow of the solution therein.

Further, the body of the pulley restrictor may be triangular-shaped and may include a recess through which the tubing is positioned. As such, the guide may include a first track and a second track nested in opposing sides of the body. The wheel may include a first projection projecting from a first side of the wheel and a second projection projecting from a second side of the wheel, where the first projection is received in the first track and the second projection is received in the second track.

In some embodiments, the enema bucket system includes a first connection at which the enema bucket is removably coupled to the tubing and a second connection at which the tubing is coupled to other tubing and the nozzle. The second connection may be provided by the anti-reflux coupler in various embodiments. Further, at least one of the first connection and the second connection is a threaded connection or an interference fit connection.

In further embodiments, the enema bucket system may include a cover configured to rest on the top of the enema bucket and cover the opening. The cover may include a filter screen, such as a mesh screen, capable of filtering at least one of coffee or tea. As such, the bucket enema system can permit use of a tea enema or a coffee enema. The bucket may include a bucket handle, wherein a hanging of the enema bucket system using the handle at a predetermined height creates water pressure that affects the speed at which the solution is expelled from the nozzle outlet.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the disclosure. Moreover, in the drawings, like reference numerals designate corresponding parts throughout the several views.

FIG. 1 is a perspective view of an enema bucket system according to various embodiments of the present disclosure.

FIG. 2 is an exploded, side elevation view of the enema bucket system according to various embodiments of the present disclosure.

FIG. 3 is a partial enlarged view of callout region 3 of FIG. 2 according to various embodiments of the present disclosure.

FIG. 4 is a partial enlarged view of callout region 4 of FIG. 2 according to various embodiments of the present disclosure.

FIG. 5 is a partial enlarged view of callout region 5 of FIG. 2 according to various embodiments of the present disclosure.

FIGS. 6A and 6B are perspective views of a pulley restrictor of the enema bucket system according to various embodiments of the present disclosure.

FIG. 7 is a partial enlarged view of a first connection of the enema bucket system according to various embodiments of the present disclosure.

FIGS. 8A and 8B are side cross-section view of an anti-reflux coupler of the bucket enema system according to various embodiments of the present disclosure.

FIG. 9 is a bottom view of a nozzle of the enema bucket system according to various embodiments of the present disclosure.

FIG. 10 is a partial enlarged view of callout region 10 of FIG. 2 showing a side cross-section view of the nozzle according to various embodiments of the present disclosure.

FIG. 11 is an enlarged cross-section view of a three-piece sleeve according to various embodiments of the present disclosure.

FIG. 12 is an exploded cross-section view of the three-piece sleeve according to various embodiments of the present disclosure.

FIG. 13 is an enlarged top view of a sealing gasket for use in the three-piece sleeve according to various embodiments of the present disclosure.

FIG. 14 is an exploded perspective view of the anti-reflux coupler according to various embodiments of the present disclosure.

FIG. 15 is a side elevation view of the enema bucket system according to various embodiments of the present disclosure.

FIG. 16 is a top view of the enema bucket system according to various embodiments of the present disclosure.

FIG. 17 is a bottom view of the enema bucket system according to various embodiments of the present disclosure.

### DETAILED DESCRIPTION

The present disclosure generally relates to an anti-reflux enema bucket system that is leakproof, easy to disassemble and clean, prevents reflux, has components that can be independently cleaned and sanitized, and controls a rate at which water is expelled from a nozzle outlet. With existing enemas, such as enemas, enema bags, and enema buckets, an opening for filling solution in the enema bag is intended to be small to prevent solution from spilling out during use, which makes filling the enema with solution difficult and inconvenient. Further, existing enemas have switches or valves that do not completely stop solution from leaking when turned off, which also leads to a buildup of dirt or other debris. Even further, various components of the enemas cannot be sterilized using, for example, boiling water, and cannot be disassembled for cleaning.

Accordingly, various embodiments are disclosed for an enema bucket system that is leakproof, easy to disassemble and clean, prevents reflux, has components that can be independently cleaned and sanitized, and controls a rate at which water is expelled from a nozzle outlet. To this end, in various embodiments, the enema bucket system includes an enema bucket having an opening at a top for filling the enema bucket with solution, a nozzle comprising a nozzle outlet, an anti-reflux coupler positioned between the enema bucket and the nozzle through which the solution passes from the enema bucket to the nozzle outlet; and tubing fluidly coupling the enema bucket, the anti-reflux coupler, and the nozzle.

The anti-reflux coupler can include an inlet for receiving at least a portion of the solution from the enema bucket; an outlet for expelling the solution into the nozzle; and a check valve configured to prevent reflux of the solution from the nozzle. The check valve may include one of an umbrella valve; a duckbill valve; a slit-cutting valve; and a flapper valve.

In some embodiments, the enema bucket system further includes a pulley restrictor configured to selectively control a speed at which the solution is introduced into the nozzle and expelled from the nozzle outlet. The pulley restrictor can include a body, a guide, and a wheel movably coupled to the guide, the body comprising a first end and a second end. The tubing can be positioned through the body of the pulley restrictor such that the wheel is positioned near the tubing. An adjustment of the wheel along the guide to the first end of the body causes pressure to be applied to the tubing and restricts flow of the solution therein. An adjustment of the wheel along the guide to the second end of the body relieves the pressure on the tubing and permits the flow of the solution therein.

Further, the body of the pulley restrictor may be triangular-shaped. In some embodiments, the body of the pulley restrictor may include a recess through which the tubing is positioned. As such, the guide comprises a first track and a second track nested in opposing sides of the body. The wheel may include a first projection projecting from a first side of the wheel and a second projection projecting from a second side of the wheel, where the first projection is received in the first track and the second projection is received in the second track.

In some embodiments, the enema bucket system includes a first connection at which the enema bucket is removably coupled to the tubing and a second connection at which the tubing is coupled to other tubing and the nozzle. The second connection may be provided by the anti-reflux coupler in various embodiments. Further, at least one of the first connection and the second connection is a threaded connection or an interference fit connection.

In further embodiments, the enema bucket system may include a cover configured to rest on the top of the enema bucket and cover the opening. The cover comprises a filter screen, such as a mesh screen, capable of filtering at least one of coffee or tea. As such, the bucket enema system can provide a tea enema or a coffee enema. The bucket may include a bucket handle, wherein a hanging of the enema bucket system using the handle at a predetermined height creates water pressure that affects the speed at which the solution is expelled from the nozzle outlet.

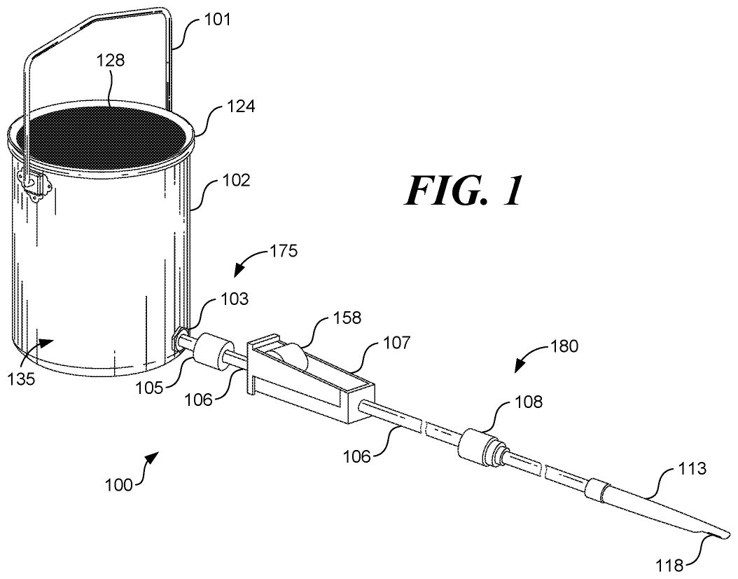

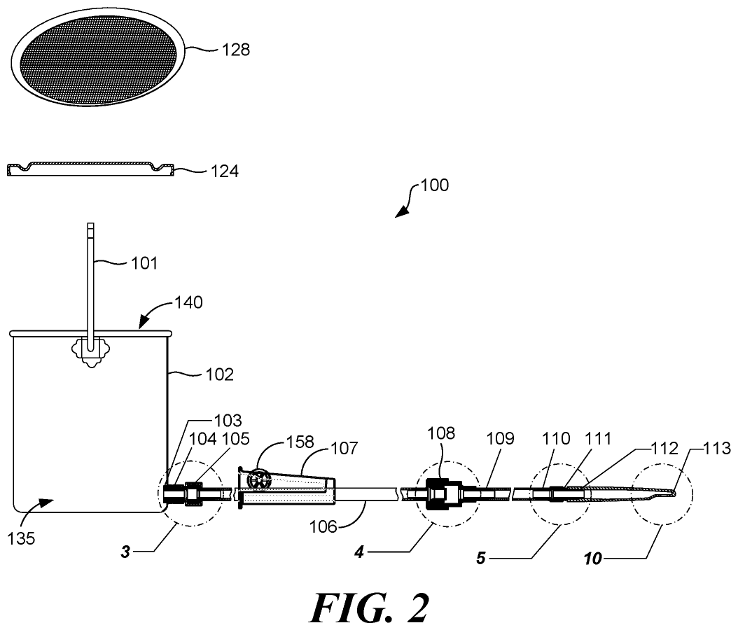

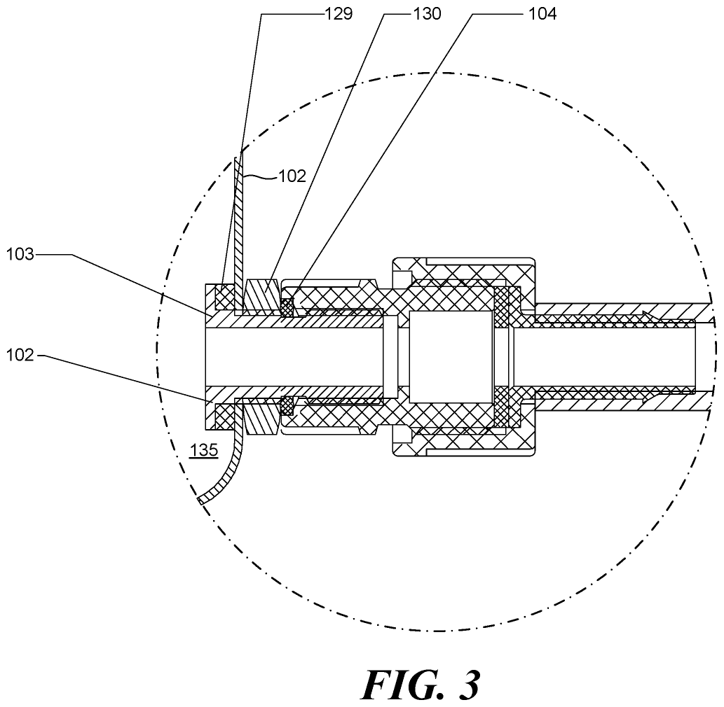

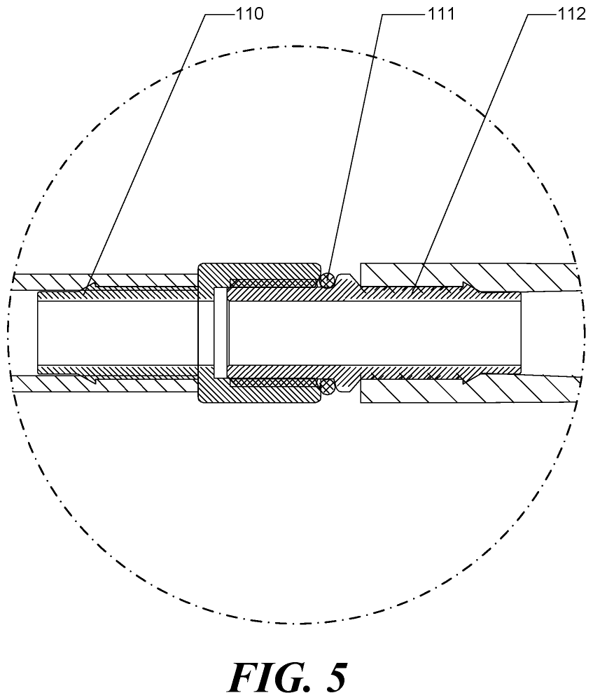

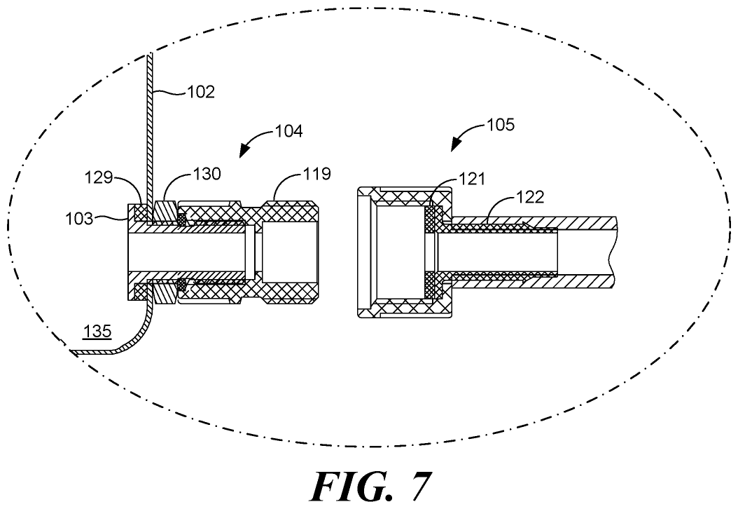

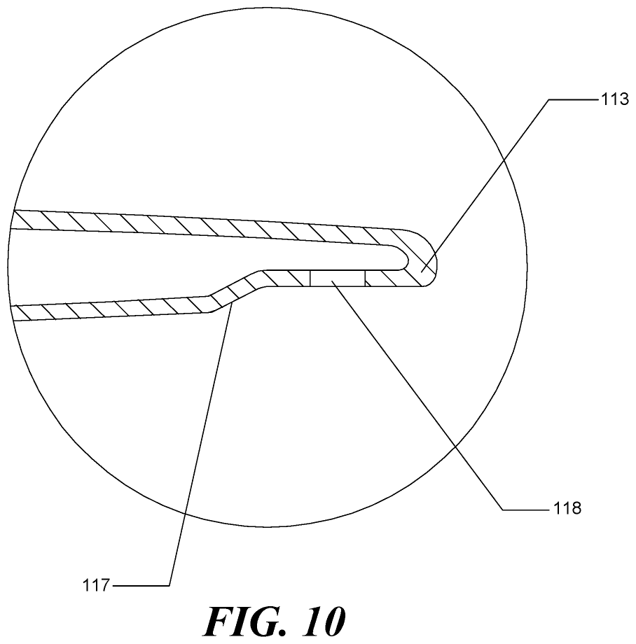

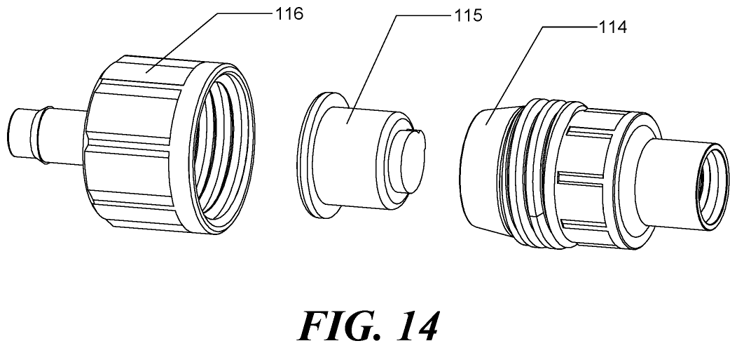

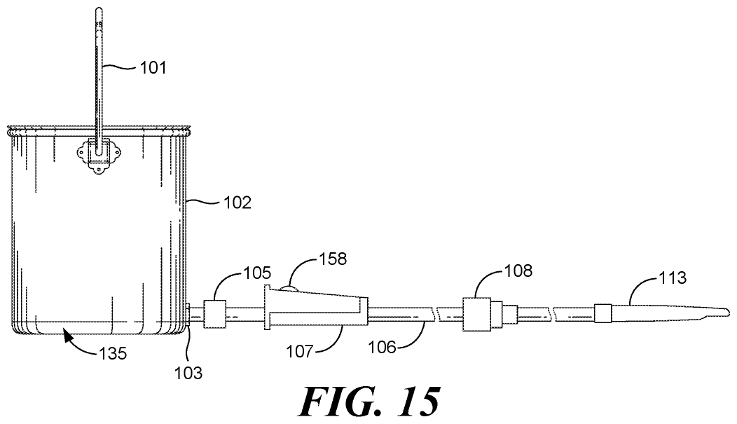

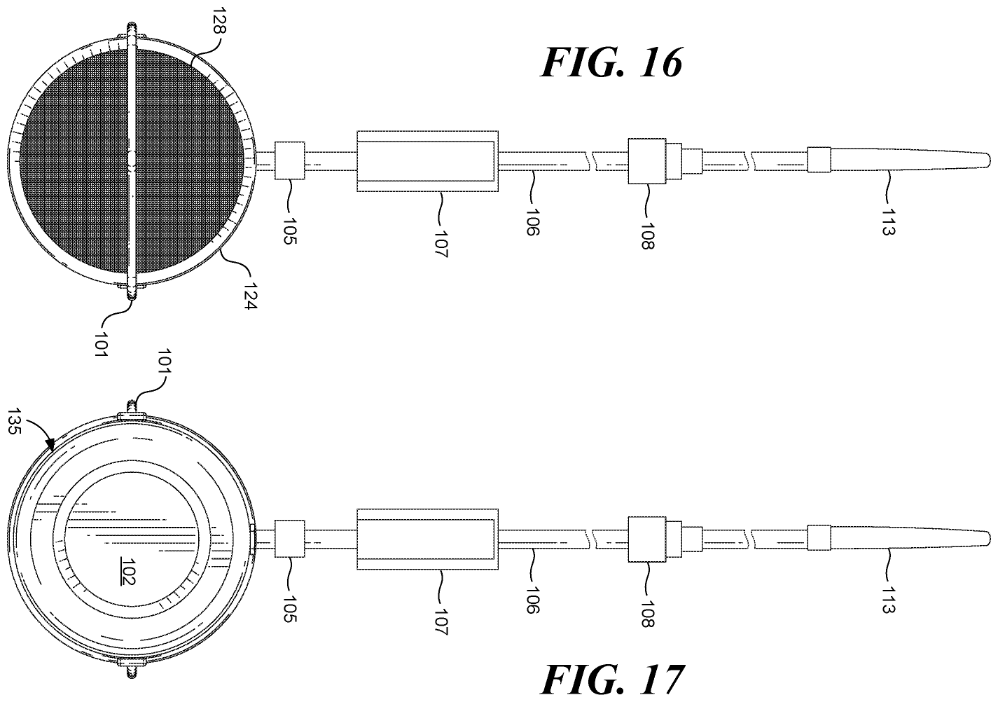

Turning now to the figures, FIGS. 1-17 illustrate various views of an enema bucket system 100 is shown according to various embodiments. Specifically, FIG. 1 is a perspective view of the enema bucket system 100 according to various embodiments. FIG. 2 is a side cross-section view of the enema bucket system 100. FIGS. 3, 4, 5, and 10 are partial, enlarged views of callout region 3, callout region 4, callout region 5, and callout region 10 of FIG. 2, respectively, according to various embodiments of the present disclosure.

Referring to FIGS. 1-17 collectively, the enema bucket system 100 may include, for example, a handle 101, an enema bucket 135 having a bucket body 102, a connector 103, a first O-ring 104, a movable three-piece sleeve 105, tubing 106, a pulley restrictor 107, an anti-reflux coupler 108, a first male connector 109, a first female connector 110, a second O-ring 111, a second male connector 112, a nozzle 113, an anti-reflux male connector 114, a one-way check valve 115, an anti-reflux female connector 116, a bevel 117, a nozzle outlet 118, an internal and external thread connector 119, a movable nut 120, a sealing gasket 121, an inner tube connector 122, an outer diameter sharp edge 123, a cover 124, a first convex 125, a second convex 126, a third convex 127, a filter screen 128, a sealing gasket 129, a metal nut 130, a bucket 135, as well as other components as will be described. In some embodiments, the first male connector 109, the first female connector 110, and the second male connector 112 can include a first M10 male connector, a first M10 female connector, and a second M10 male connector, respectively, or other suitable type of connector.

More specifically, the enema bucket system 100 may include an enema bucket 135 and a handle 101. The handle 101 may be positioned on an upper part of the enema bucket 135. In some embodiments, the handle 101 has a pointed horizontal cross-member connecting each vertical arm of the handle 101. A cover 124 may be provided below the handle 101, for instance, between each arm of the handle 101 to rest on a top surface of the bucket body 102. The bottom of the handle 101 may be coupled to an enema bucket 135 or, more specifically, to an upper portion of a bucket body 102. A connector 103, such as a metal connector or other suitable connector, may be positioned at a lower portion of the bucket body 102 to couple to tubing 106 through which solution passes on its way to the nozzle 113. As such, all of the tubing 106 and any components coupled thereto, as well be described, may be removed via the connector 103.

In various embodiments, a movable three-piece sleeve 105 may be provided beside or near the connector 103, and a first O-ring 104 may be provided between the connector 103 and the movable three-piece sleeve 105. The first O-ring 104 and the movable three-piece sleeve 105 may form a seal, as may be appreciated, and the movable three-piece sleeve 105 may be tightly coupled to tubing 106 at the other end. The tubing 106 may include silicone tubing in some embodiments. Further, the tubing 106 may be coupled to or otherwise connected with a pulley restrictor 107. The pulley restrictor 107 may be configured to control a wheel in a pulley-like fashion to adjust, stop, or start a speed at which solution flows through the tubing 106 and/or out the nozzle outlet 118, as will be described in greater detail below.

The tubing 106, for instance, as coupled to the pulley restrictor 107 may be provided with an anti-reflux coupler 108 that is removable and washable. The anti-reflux coupler 108 may be connected or otherwise fixed to the tubing 106 using a straight and closed-type fitting. In some embodiments, the tubing 106 forms an interference fit, a threaded connection fit, or other suitable fit with ends of the anti-reflux coupler 108. For example, the anti-reflux coupler 108 may have an end adopting an external thread to connect with a first male connector 109. The first male connector 109 may be provided with an O-ring, and the O-ring and the first male connector 109 may form a sealed connection. An end of the tubing 106 may be sealed with the first female connector 110 in a closed-fitting fashion. The second male connector 112 may be positioned on a right side of the first female connector 110. In some embodiments, the first male connector 109 has a second O-ring 111. The second male connector 112 may be connected to the nozzle 113, which may include a straight enema mouth in some embodiments, where the side of the straight enema mouth may be provided with the nozzle outlet 118.

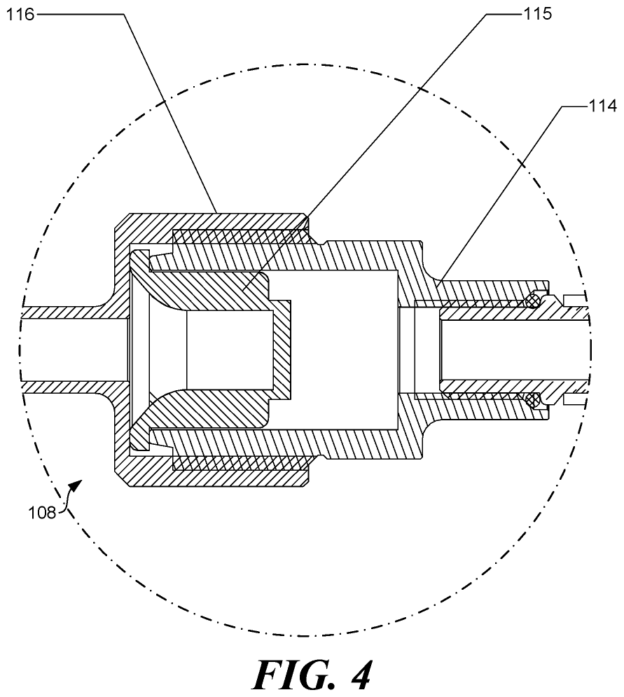

Referring now to FIG. 4, an enlarged view of the anti-reflux coupler 108 is shown. In various embodiments, the anti-reflux coupler 108 may include various components, such as an anti-reflux male connector 114, a one-way check valve 115 (or check valve 115), and an anti-reflux female connector 116. The check valve 115 can include one of an umbrella valve, a duckbill valve, a slit-cutting valve, and a flapper valve in some embodiments. In some embodiments, the check valve 115 is a silicone check valve. The anti-reflux female connector 116 and the anti-reflux male connector 114 may be threaded and the check valve 115 may be set in the anti-reflux male connector 114 in some embodiments. As such, the anti-reflux coupler 108 is a device having threaded connections, detachable, provides a strong sealing effect, washable, durable, and is capable of repeated high temperature sterilization.

In various embodiments, the enema bucket 135 and the handle 101 are formed of stainless steel, such as medical grade 304 stainless steel material. As such, the enema bucket 135 and/or the handle 101 are firm and solid, and provide a smooth handle surface free of burrs that occur commonly in manufactured plastic. The handle 101 may include a curbed or sharp-pointed top portion in the horizontal cross-member that is convenient for an operator to hang the enema bucket system 100 on a door hook in a secure manner, or in another suitable location.

In some embodiments, the enema bucket system 100 includes a cover 124 for positioning above a bucket mouth 140 and preventing solution from being spilled during operation. In some embodiments, the cover 124 is made of soft polyethylene (PE), and a vent hole (not shown) may be provided on the cover 124 permit filling of solution while not affecting the operation of the enema bucket system 100. The cover 124 may be an anti-impact material, and can be repeatedly boiled and sterilized. In some embodiments, the cover 124 is sized and positioned to cover the entirety of the bucket mouth 140. The cover 124 may include sealing edges that form a friction fit and/or interference fit with the bucket mouth 140 or a top rip of the enema bucket 135. When an enema is being performed, dirt and other debris can be effectively prevented from falling into the enema bucket 135. When water or other solution is added to an interior of the enema bucket 135, the cover 124 can be easily removed.

Further, the bucket body 102 may equipped with a filter screen 128. The filter screen 128 can permit coffee enemas, tea enema, and other enema in which filtration is beneficial. For instance, coffee grinds, tea bags, tea leaves, or other item can be placed on the filter screen 128 and water added to create a liquid solution that resides in the interior of the enema bucket 135. Alternatively, the filter screen 128 can prevent debris from entering the enema if and when solution is continuously added. In some embodiments, the filter screen 128 is formed of medical grade 304 stainless steel for filtering items, such as coffee or tea. The filter screen 128 may include a size slightly less than the cover 124 such that the filter screen 128 is covered on the bucket mouth 140 and is not easily loosened, while being easy to take off for cleaning. Also, the filter screen 128 may be boiled and sterilized and reused repeatedly.

Similarly, the bucket body 102 and/or other components of the enema bucket 135 may be formed of medical grade 304 stainless steel material, which is resilient and not easily broken. The bucket body 102 may be repeatedly boiled and sterilized while providing good durability. Meanwhile, the bucket mouth 140 is configured to have a wide mouth that facilitates filling of the enema bucket 135 with solution, and it is also convenient for the operator to thoroughly clean and sterilize before or after use.

A lower portion, such as a distal lower portion, of the bucket body 102 may be provided with a connector 103, such as a metal connector. The connector 103 may be closely fit to the bucket body 102 and sealed with a gasket. A portion of the connector 103 being positioned outside of the bucket body 102 may be firmly connected with one or more nuts. If an operator moves the enema bucket 135 during use, the enema bucket 135 and the tubing 106 do not easily to loosen or leak. The connector 103 may include an external thread structure, and the external thread structure may be sleeved with an O-ring 104, such as a silicone O-ring, which forms a tight seal with the internal thread of the movable three-piece sleeve 105. As such, water leakage does not easily occur when fastening. Moreover, it is convenient for the operator to attach and detach the tubing 106 and other components of the enema bucket system 100 for cleaning, sterilization, or storage.

The movable three-piece sleeve 105 in the enema bucket system 100 may be made of medical grade polypropylene (PP) plastic. As such, the movable three-piece sleeve 105 can be disassembled and cleaned, such as through boiling and sterilization at high temperatures. The movable three-piece sleeve 105 may include a silicone gasket that provides a strong sealing effect where water leakage does not easily occur. In some embodiments, a first end of the movable three-piece sleeve 105 is designed to include an internal thread and an external thread with an O-ring metal connector. The thread-type tight sealing connection is firm, and leakage does not easily occur. The second end of the movable three-piece sleeve 105 may include a straight inverted hook that is tightly sealed with the tubing 106, providing a firm connection that does not easily loosen. The movable three-piece sleeve 105 is movable due to the presence of an internal threaded cap. When the operator attaches and detaches the tubing 106 and other components the bucket body 102, the tubing 106 will not be twisted and easily entangled, which is convenient for the operator to easily mount and disassemble and clean the tubing 106 through sterilization.

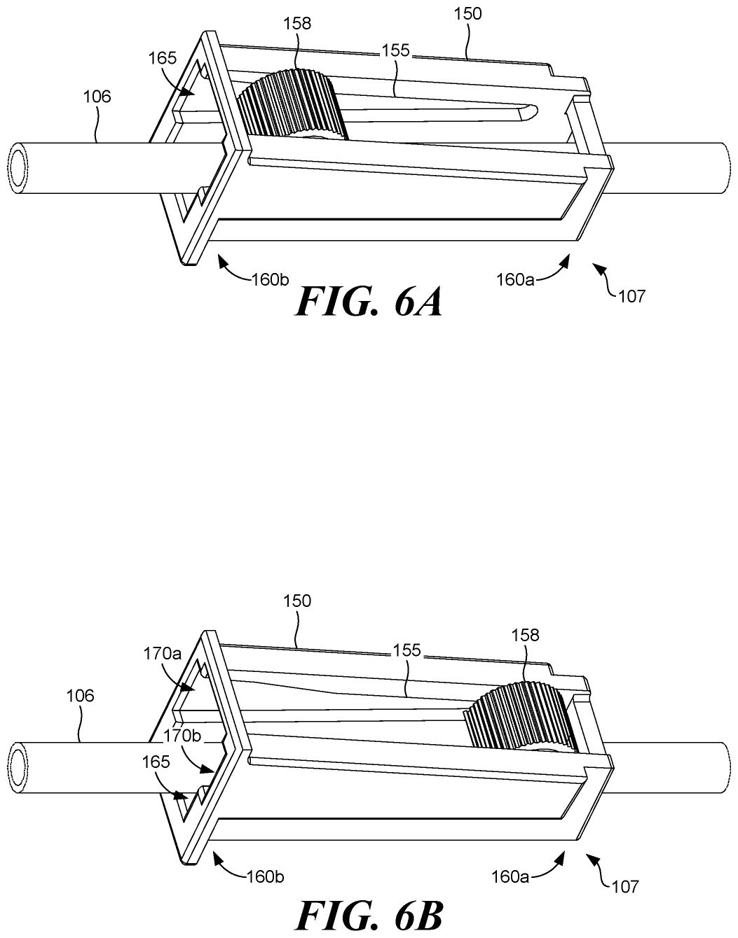

Turning now to FIGS. 6A and 6B, various views of the pulley restrictor 107 of the enema bucket system 100 are shown according to various embodiments of the present disclosure. As noted above, in various embodiments, the enema bucket system 100 can include a pulley restrictor 107 configured to selectively control a speed at which the solution is introduced into the nozzle 113 and expelled from the nozzle outlet 118. The pulley restrictor 107 can include a body 150 (or “pulley restrictor body”), a guide 155, and a wheel 158 movably coupled to the guide 155, where the body 150 has a first end 160a and a second end 160b. The tubing 106 can be positioned through an aperture 165 of the body such that the wheel 158 is positioned above, below, to the side of, or otherwise near the tubing 106. The guide 155 is angled downwards in some example with causes a relative vertical and horizontal position of the wheel 158 to adjust, for instance, through rotation of the wheel 158 using a thumb or finger of the operator which can cause the wheel 158 to come into contact with the tubing 106.

As shown between the views of FIGS. 6A and 6B, an adjustment of the wheel 158 along the guide 155 to the first end 160a of the body 150 (e.g., rotating the wheel 158 in a first or second horizontal direction) causes a vertical position of the wheel 158 relative to the tubing 106 to move downwards and a horizontal position of the wheel 158 to move rightwards, causing the wheel 158 to apply pressure to a surface of the tubing 106, thereby restricting flow of any solution therein. Conversely, adjustment of the wheel 158 along the guide 155 to the second end 160b of the body 150 relieves the pressure on the tubing 106 and permits the flow of the solution therein. In other words, when the wheel 158 is moved to the second end 160b of the body 150, the wheel 158 has no or negligible contact with the tubing 106.

The body 150 of the pulley restrictor 107 can be triangular-shaped. Further, the body 150 of the pulley restrictor 107 may comprise an aperture 165 through which the tubing 106 is positioned. The guide 155 can include a first track 170a and a second track 170b nested in opposing sides of the body 150 of the pulley restrictor 107. As such, the wheel 158 can include a first projection (not shown) projecting from a first side of the wheel 158 and a second projection (not shown) projecting from a second side of the wheel 158, where the first projection is received in the first track 170a and the second projection is received in the second track 170b, as may be appreciated.

In some embodiments, the pulley restrictor 107 made of medical grade polypropene (PP) plastic. Thus, when the pulley restrictor 107 is removed from the tubing 106, the pulley restrictor 107 can be boiled at high temperature for sterilization before or after use. Notably, the pulley restrictor 107 has a smooth surface and is free of burrs. The pulley restrictor 107 has a unique shape and a serration design. The sliding pulley position or, in other words, the position of the wheel 158, can be used to clamp the tubing 106 to adjust the flow of solution in the tubing 106. The pulley restrictor 107 may be sleeved outside the tubing 106 and, as such, does not create connections at which leaks occur while still being convenient for mounting and disassembling, and cleaning and sterilization. The pulley restrictor 107 is sized and positioned to be convenient for the operator to grasp with a single hand and easily operate the position of the wheel 158 to adjust a speed or flow of solution, thereby increasing comfort. When the operator wants to stop the enema, the position of the wheel 158 in the pulley restrictor 107 can be easily adjusted with a thumb or other finger to stop the flow.

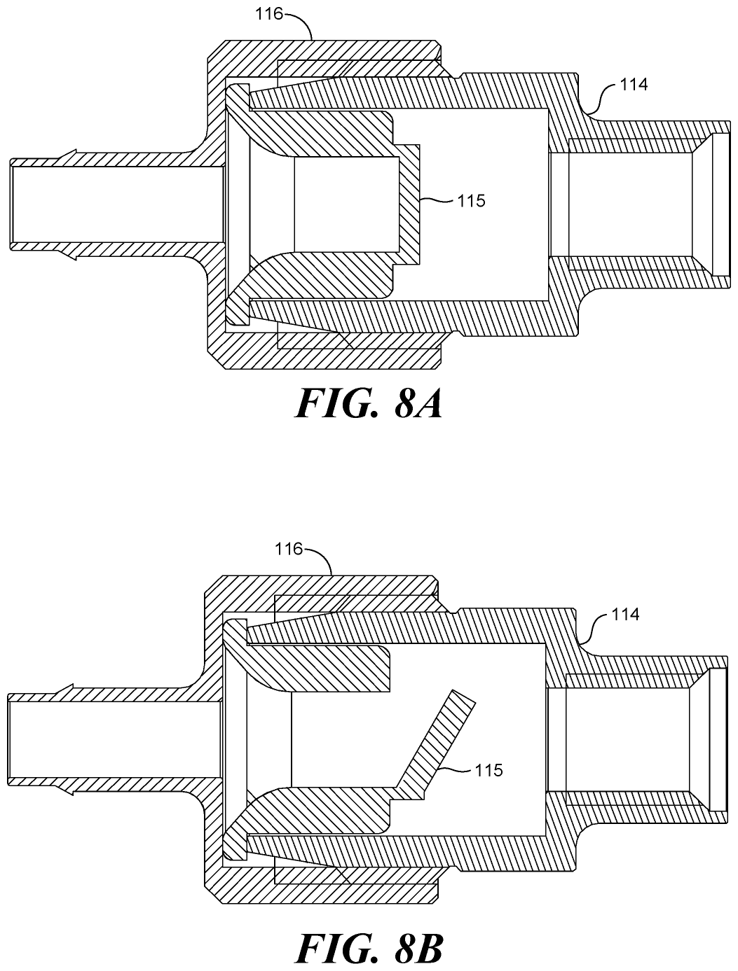

In various embodiments, the anti-reflux coupler 108 may be composed of three components, such as the anti-reflux male connector 114, the check valve 115, and the anti-reflux female connector 116. These fittings may be connected through a threaded connection, as shown in FIGS. 8A and 8B, which provides a good sealing effect and is convenient for the patient to repeatedly open and clean, sterilize under a high temperature, and has strong durability. The anti-reflux coupler 108 has a strong anti-reflux effect, and sewage or solution refluxing from a bodily cavity does not easily renter the tubing 106 or other component of the enema bucket system 100. At the same time, a large amount of outflow of solution from the check valve 115 will not cause leakage and sewage reflux, thus saving solution.

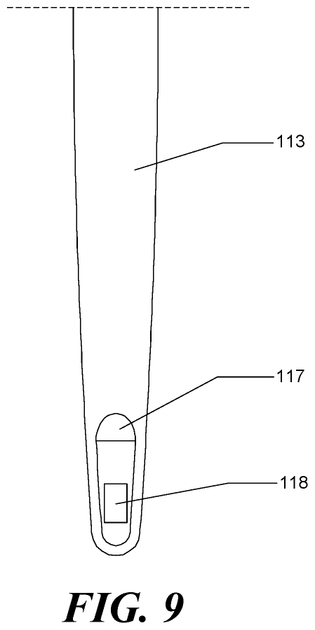

Referring next to FIG. 9, a bottom view of the nozzle 113 of the enema bucket system 100 is shown according to various embodiments of the present disclosure. The nozzle 113 may include a straight enema tip mouth made of medically soft materials, which is also environmentally friendly and durable. In some embodiments, an entirety of the straight enema tip mouth is smooth and burr-free without having a joint line that can cause cuts or other discomforts to the operator. The head of the nozzle 113 may be straight, and the nozzle outlet 118 may be positioned at the side of the nozzle 113. The nozzle outlet 118 may have a large mouth that provides a large flow of water or other solution. The straight face enema tip mouth, due to straight head design, may include a one-sided flat design, as depicted in FIG. 9. Specifically, the nozzle outlet 118 may be positioned on a flat size of the nozzle 113. As such, after entering a bodily cavity, the nozzle outlet 118 is less likely to be obstructed as compared to an ordinary outer surface hole. The nozzle 113 may include a bevel 117 positioned below the nozzle outlet 118 as a transitioning design, which facilitates the nozzle 113 smoothly entering a bodily cavity of the human body, and the surface of the washing head has no joint line and no edges and corners. The nozzle outlet 118 is set on the plane, where the injection mold is a plane touch mold, avoiding burrs and flashing, which are known to scratch the human body. After entering the body, the nozzle 113 will not cause a sting feeling and, at the same time, will not block the nozzle outlet 118.

In various embodiments, each of the components of the enema bucket system 100 can be formed of at least one of medical grade: stainless steel, polyethylene plastic, polypropylene plastic, or other suitable material. As such, each of the components of the enema bucket system 100 can be sterilized by boiling water.

In this case, the nozzle 113 coupled to the tubing of the movable three-piece may be removed from the tubing 106 before use, and then the remaining tubing 106 and enema bucket 135 may be placed into boiling water for 5-10 minutes to sterilize. After boiling the components to sterilize, the components may be removed, and the tubing 106 and the enema bucket 135 may be dried. The movable three-piece is then coupled to the connector 103 of the enema bucket 135. Then, the nozzle 113 may be coupled at the M10 female connector 110. The wheel 158 of the pulley restrictor 107 may be adjusted to the closed state. Warm enema water or other solution may be prepared and poured into the enema bucket 135 via the bucket mouth 140. Then, the enema bucket system 100 may be suspended as filled with solution to a desired height. Thereafter, the operator may slowly adjust the pulley restrictor 107 to an open state.

The flow rate may be adjusted to be as small as possible until any air in the tubing 106 is exhausted. In a state of natural high suspension, the solution may have a certain pressure to jack the check valve 115 of the anti-reflux coupler 108 open (as shown in FIG. 8B), thereby causing solution to flow through the tubing 106 to nozzle 113 to produce water discharge. Then, the wheel 158 of the pulley restrictor 107 may be adjusted to a closed state. The nozzle 113 may be coated with a lubricant, such as organic lubricating oil, and the nozzle 113 may be inserted into a bodily cavity. The wheel 158 of the pulley restrictor 107 may be adjusted to an open state to start the enema process where solution is discharged from the nozzle outlet 118. During the enema process, due to certain pressure arising in the human body, sewage will be refluxed to the tubing 106. At this time, the check valve 115 will be completely closed (as shown in FIG. 8A) when the check valve 115 in the anti-reflux coupler 108 is subject to a counter force, thus sewage will not reflux beyond the anti-reflux coupler 108. Accordingly, the enema bucket system 100 includes threaded connections, both having sealing rings (e.g., O-ring seals), a strong sealing effect is provided without leakage of solution. As such, waste of solution does not occur.

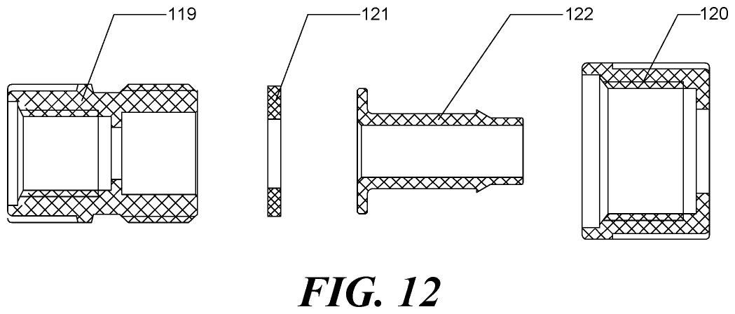

FIG. 12 is an exploded cross-section view of the movable three-piece sleeve 105 according to various embodiments of the present disclosure. Operation of the movable three-piece sleeve 105 is now described. One hand of the operator may pinch an anti-skid rib of an internal and external thread connector 119, and the other hand may loosen the movable nut 120 counterclockwise to let the movable nut 120 loosen from the external thread of the internal and external thread connector 119. Due to the movable nut 120 and the inner tube connector 122 being separated, when the movable nut 120 rotates, the inner tube connector 122 does not rotate therewith. As such, the tubing 106 does not rotate or twist during removal.

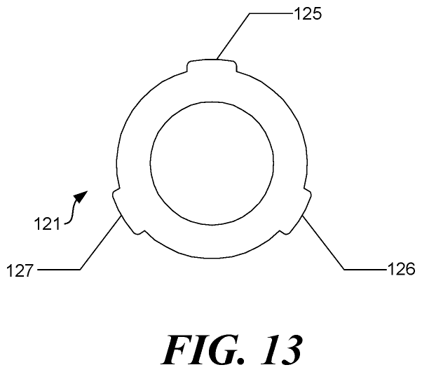

FIG. 13 is an enlarged top view of the sealing gasket 121 according to various embodiments of the present disclosure. The sealing gasket 121 may be formed of a silicone material that provides a good sealing effect. The outer ring of the sealing gasket 121 may be provided with a first convex 125, a second convex 126, and a third convex 127 to prevent the sealing gasket 121 from coming out of the movable nut 120.

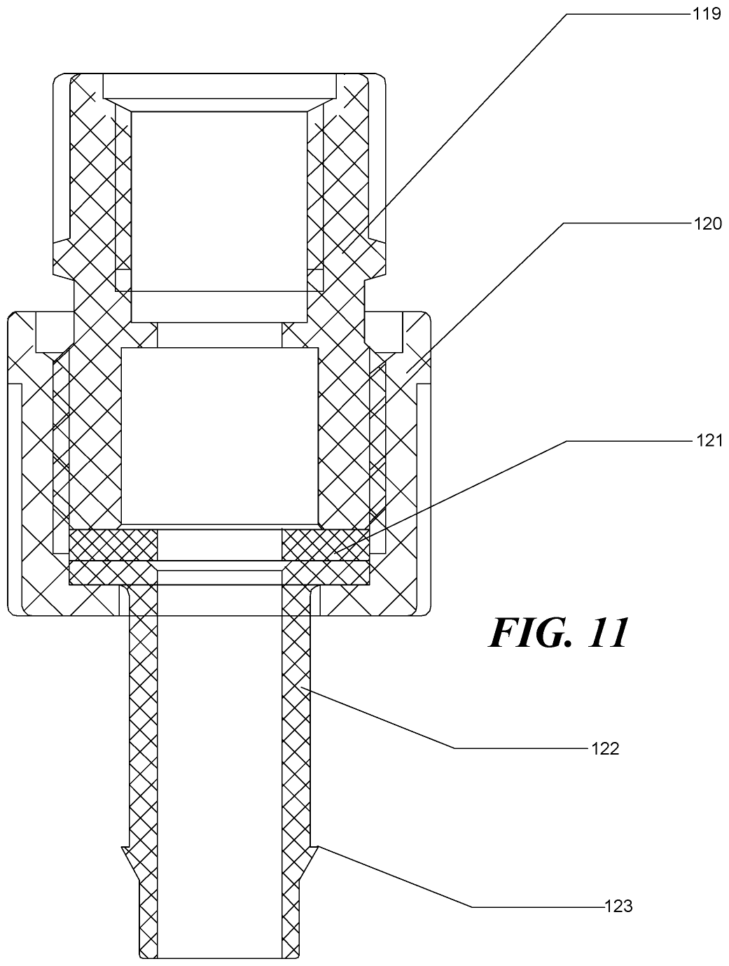

Referring again to FIG. 11, a lower portion of the inner tube connector 122 may provided with an outer diameter sharp edge 123, which is processed into a unique sharp angle by a precision mold mirror pulse to form a tight sealing connection and a vacuum when inserted straightly into the tubing 106, thereby ensuring that the connection does not leak solution, and the sharp corner inverted manner can also effectively prevent the tubing from coming out of the inner tube connector, providing a very firm connection.

Referring again to FIGS. 1-3, the enema bucket system 100 can further include a first connection 175 at which the enema bucket 135 is removably coupled to the tubing 106, and a second connection 180 at which the tubing 106 is coupled to other tubing and the nozzle 113. In some embodiments, the second connection 180 is provided via the anti-reflux coupler 108. At least one of the first connection 175 and the second connection 180 is a threaded connection or an interference fit connection.

As noted above, the tubing 106 is provided with a pulley restrictor 107, which is convenient for an operator to easily adjust toggle the solution (e.g., on or off) as well as the speed of the solution with a single hand, enhancing comfort when using the enema bucket system 100. The anti-reflux coupler 108 has a good flow rate, and the valve has a good anti-reflux effect. In some embodiments, the anti-reflux coupler 108 can be referred to as washable anti-reflux duckbilled one-way valve. As such, reflux of solution is avoided, and the anti-reflux coupler 108 can be separately disassembled and disinfected.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, the person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc., unless otherwise specified in the appended claims. The terms “first,” “second,” etc. are used only as labels, rather than a limitation for a number of the objects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. An enema bucket system, comprising:
an enema bucket having an opening at a top for filling the enema bucket with solution;
a nozzle comprising a nozzle outlet;
an anti-reflux coupler positioned between the enema bucket and the nozzle through which the solution passes from the enema bucket to the nozzle outlet, the anti-reflux coupler comprising:
an inlet for receiving at least a portion of the solution from the enema bucket;
an outlet for expelling the solution into the nozzle; and
a check valve configured to prevent reflux of the solution from the nozzle;
tubing fluidly coupling the enema bucket, the anti-reflux coupler, and the nozzle; and
a pulley restrictor configured to selectively control a speed at which the solution is introduced into the nozzle and expelled from the nozzle outlet.

2. The enema bucket system of claim 1, wherein the bucket comprises a bucket handle, wherein a hanging of the enema bucket system using the handle at a predetermined height creates water pressure that affects the speed at which the solution is expelled from the nozzle outlet.

3. The enema bucket system of claim 1, wherein:
the pulley restrictor comprises a body, a guide, and a wheel movably coupled to the guide, the body comprising a first end and a second end;
the tubing is positioned through the body such that the wheel is positioned near the tubing;
adjustment of the wheel along the guide to the first end of the body causes pressure to be applied to the tubing and restricts flow of the solution therein; and
adjustment of the wheel along the guide to the second end of the body relieves the pressure on the tubing and permits the flow of the solution therein.

4. The enema bucket system of claim 3, wherein:
the body of the pulley restrictor is triangular-shaped and comprises a recess through which the tubing is positioned;
the guide comprises a first track and a second track nested in opposing sides of the body;
the wheel comprises a first projection projecting from a first side of the wheel and a second projection projecting from a second side of the wheel; and
the first projection is received in the first track and the second projection is received in the second track.

5. The enema bucket system of claim 1, wherein the check valve comprises one of: an umbrella valve; a duckbill valve; a slit-cutting valve; and a flapper valve.

6. The enema bucket system of claim 1, further comprising:
a first connection at which the enema bucket is removably coupled to the tubing; and
a second connection at which the tubing is coupled to other tubing and the nozzle, the second connection being provided by the anti-reflux coupler.

7. The enema bucket system of claim 6, wherein at least one of the first connection and the second connection is a threaded connection or an interference fit connection.

8. An enema bucket system, comprising:
an enema bucket;
a nozzle comprising a nozzle outlet;
an anti-reflux coupler positioned between the enema bucket and the nozzle through which the solution passes from the enema bucket to the nozzle outlet, the anti-reflux coupler comprising:
an inlet for receiving at least a portion of the solution from the enema bucket;
an outlet for expelling the solution into the nozzle; and
a check valve configured to prevent reflux of the solution from the nozzle; and
tubing fluidly coupling the enema bucket, the anti-reflux coupler, and the nozzle.

9. The enema bucket system of claim 8, wherein:
the enema bucket comprises an opening at a top of the enema bucket for filling the enema bucket with solution; and
the enema bucket system further comprises a cover configured to rest on the top of the enema bucket and cover the opening.

10. The enema bucket system of claim 9, wherein the cover comprises a filter screen.

11. The enema bucket system of claim 8, wherein the bucket comprises a bucket handle, wherein a hanging of the enema bucket system using the handle at a predetermined height creates water pressure that affects the speed at which the solution is expelled from the nozzle outlet.

12. The enema bucket system of claim 9, further comprising a pulley restrictor configured to selectively control a speed at which the solution is introduced into the nozzle and expelled from the nozzle outlet.

13. The enema bucket system of claim 12, wherein:
the pulley restrictor comprises a body, a guide, and a wheel movably coupled to the guide, the body comprising a first end and a second end;
the tubing is positioned through the body such that the wheel is positioned near the tubing;
adjustment of the wheel along the guide to the first end of the body causes pressure to be applied to the tubing and restricts flow of the solution therein; and
adjustment of the wheel along the guide to the second end of the body relieves the pressure on the tubing and permits the flow of the solution therein.

14. The enema bucket system of claim 13, wherein:
the body of the pulley restrictor is triangular-shaped and comprises a recess through which the tubing is positioned;
the guide comprises a first track and a second track nested in opposing sides of the body;
the wheel comprises a first projection projecting from a first side of the wheel and a second projection projecting from a second side of the wheel; and
the first projection is received in the first track and the second projection is received in the second track.

15. The enema bucket system of claim 9, wherein the check valve comprises one of: an umbrella valve; a duckbill valve; a slit-cutting valve; and a flapper valve.

16. The enema bucket system of claim 9, further comprising:
a first connection at which the enema bag is removably coupled to the tubing; and
a second connection at which the tubing is coupled to other tubing and the nozzle, the second connection being provided by the anti-reflux coupler.

17. The enema bucket system of claim 9, wherein at least one of the first connection and the second connection is a threaded connection or an interference fit connection.

18. A method, comprising:
providing an enema bucket system, comprising:
an enema bucket;
a nozzle comprising a nozzle outlet;
an anti-reflux coupler positioned between the enema bucket and the nozzle through which the solution passes from the enema bucket to the nozzle outlet, the anti-reflux coupler comprising:
an inlet for receiving at least a portion of the solution from the enema bag;
an outlet for expelling the solution into the nozzle; and
a check valve configured to prevent reflux of the solution from the nozzle; and
tubing fluidly coupling the enema bucket, the anti-reflux coupler, and the nozzle.

19. The method of claim 18, wherein the enema bucket system further comprises a pulley restrictor configured to selectively control a speed at which the solution is introduced into the nozzle and expelled from the nozzle outlet.

20. The method of claim 19, wherein:
the pulley restrictor comprises a body, a guide, and a wheel movably coupled to the guide, the body comprising a first end and a second end;
the tubing is positioned through the body such that the wheel is positioned near the tubing;
adjustment of the wheel along the guide to the first end of the body causes pressure to be applied to the tubing and restricts flow of the solution therein;
adjustment of the wheel along the guide to the second end of the body relieves the pressure on the tubing and permits the flow of the solution therein;
the body of the pulley restrictor is triangular-shaped and comprises a recess through which the tubing is positioned;
the guide comprises a first track and a second track nested in opposing sides of the body;
the wheel comprises a first projection projecting from a first side of the wheel and a second projection projecting from a second side of the wheel; and
the first projection is received in the first track and the second projection is received in the second track.
